(19) United States
(12) Patent Application Publication
(10) Pub. No.: US 2014/0056987 A1
(43) Pub. Date: Feb. 27, 2014

(54) COMPOSITIONS COMPRISING EXTRACTS OR FRACTIONS DERIVED FROM ANNONA SQUAMOSA FOR THE PREVENTION, TREATMENT OR CONTROL OF INFLAMMATORY AND METABOLIC DISORDERS

(71) Applicant: LAILA NUTRACEUTICALS, Vijayawada (IN)

(72) Inventors: Ganga Raju GOKARAJU, Vijayawada (IN); Rama Raju Gokaraju, Vijayawada (IN); Venkata Kanaka Ranga Raju Gokaraju, Vijayawada (IN); Trimurtulu Golakoti, Vijayawada (IN); Kiran Bhupthiraju, Vijayawada (IN); Venkata Krishna Raju Alluri, Vijayawada (IN); Krishanu Sengupta, Vijayawada (IN)

(73) Assignee: LAILA NUTRACEUTICALS, Vijayawada (IN)

(21) Appl. No.: 14/065,583

(22) Filed: Oct. 29, 2013

Related U.S. Application Data
(63) Continuation-in-part of application No. PCT/IN2012/000313, filed on Apr. 27, 2012.
Application No. PCT/IN2012/000313 is a continuation-in-part of application No. 13/450,124, filed on Apr. 18, 2012.

(30) Foreign Application Priority Data
Apr. 29, 2011 (IN) 1482/CHE/2011

(51) Int. Cl. A61K 36/185; A61K 36/9066; A61K 36/19; A61K 31/202; A61K 31/355; A61K 31/198; A61K 36/324; A61K 31/375

(52) U.S. Cl. CPC A61K 36/185; A61K 36/324; A61K 36/9066; A61K 31/375; A61K 31/202; A61K 31/355; A61K 31/198; A61K 36/19
USPC 424/522; 424/725; 424/702; 424/769; 424/756

(57) ABSTRACT

The invention discloses synergistic composition comprising at least one Annona squamosa derived component standardized to acetogenin compound(s) having α,β-unsaturated-γ-methyl-γ-lactone moiety in combination with at least one biologically active ingredient derived from plants, animals or microorganisms such as vitamins, amino acids, minerals, fibers, various plant and herbal extracts for the prevention, treatment, inhibition or controlling inflammation and/or metabolic disorders and other associated or related diseases. The invention further discloses the method of treating inflammation and/or metabolic disorders and other associated or related diseases.

### RELATED APPLICATION

This application is a continuation-in-part of U.S. patent application Ser. No. 13/450,124, filed Apr. 18, 2012 and published as U.S. Patent Publication 2012/0201884, which is a continuation-in-part of International Application PCT/1N2010/000686, filed on Oct. 19, 2010. This application is also a continuation of International Application PCT/IN2012/000313, filed on Apr. 27, 2012 and published as International Patent Publication WO 2012/147106. The entire disclosure of each prior application is hereby incorporated by reference in its entirety.

### FIELD OF INVENTION

The invention relates to synergistic composition comprising at least one Annona squamosa derived component selected from extract, fraction, compound or mixtures thereof in combination with at least one biologically active ingredient derived from plants, animals or microorganisms such as vitamins, amino acids, minerals, fibers, various plant and herbal extracts. The synergistic compositions can be useful for the prevention, control and/or treatment of inflammation and/or metabolic disorders.

### BACKGROUND OF THE INVENTION

Annona squamosa, a small evergreen tree, is cultivated throughout India for its fruits. Different parts of Annona squamosa are used in folklore medicine for the treatment of several disorders including cancer, cardiac diseases, diabetes and hyperthyroidism. Fruits are normally eaten fresh. Between 50-80% of the fruit is edible. The few ent-kaurane compounds isolated from the stems of Annona squamosa exhibited immunomodulating effects in leukocytes. Caryophyllene oxide was isolated from an unsaponified petroleum ether extract of the bark of Annona squamosa and studied for its analgesic and anti-inflammatory activity. Two new cyclic peptides, cyclosquamosin H and I together with six known cyclic peptides, squamin A (3), squamin B (4), cyclosquamosin A (6), cyclosquamosin D (7), cyclosquamosin E (8), and cherimolacyclopeptide B (9) have been disclosed. [Yang Y L et al., J Agric Food Chem. 2008 Jan. 23; 56(2):386-92. Epub 2007 Dec. 12].

The natural antidotes for free radicals are antioxidants, which include vitamins C and E and the flavonoids found in vegetables, fruits and herbs. Many antioxidants directly counteract the pro-inflammatory effects of free radicals. Vitamin C is a useful antioxidant nutrient. Indeed, humans' inability to convert blood sugar to vitamin C may very well predispose people to type-2 diabetes because excess glucose remains in the blood instead of being converted to vitamin C. Because diabetics have higher levels of CRP and IL-6, the markers of inflammation, increasing vitamin C levels can modulate both blood sugar spikes as well as its attendant inflammation. One report found that people with peripheral arterial disease were more likely to have greater inflammation and severe heart disease when their blood levels of vitamin C were low.

Because the cause of chronic inflammation is largely nutritional, nutrition is the best way to reverse it. Vitamins E and C, the omegas, flavonoids, and other supplements are well documented for their roles in correcting pro-inflammatory nutritional imbalances and for reducing inflammation.

It is also estimated that about 64% of Americans are overweight or obese (roughly about 97 million adults) and it is generally believed that these numbers are increasing. Being obese or overweight may lead to several problems such as substantially increased the risk of morbidity from hypertension; dyslipidemia; type 2 diabetes; coronary heart disease; stroke; gallbladder disease; osteoarthritis; sleep apnea and respiratory problems; and endometrial, breast, prostate, and colon cancers.

For these reasons, there is an enormous interest in treating metabolic disorders such as obesity. Existing therapies of obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery, such as gastric bypass. Caloric restriction, regardless of its form, can cause catabolism of body protein and produce negative nitrogen balance.

In view of the foregoing there remains a need for effective compositions for preventing, controlling and/or treating inflammation and several inflammation associated diseases and/or for controlling, preventing or treating obesity and metabolic disorders and associated conditions and disorders. The present invention thus provides synergistic compositions which are effective against inflammation and/or metabolic disorders.

### SUMMARY OF THE INVENTION

The invention discloses synergistic composition comprising at least one Annona squamosa derived component standardized to acetogenin compound(s) having α,β-unsaturated-γ-methyl-γ-lactone moiety in combination with at least one biologically active ingredient derived from plants, animals or microorganisms such as vitamins, amino acids, minerals, fibers, various plant and herbal extracts.

The invention discloses synergistic composition comprising at least one Annona squamosa derived component standardized to acetogenin compound(s) having α,β-unsaturated-γ-methyl-γ-lactone moiety in combination with at least one biologically active ingredient derived from plants, animals or microorganisms such as vitamins, amino acids, minerals, fibers, various plant and herbal extracts for the prevention, treatment, inhibition or controlling inflammation and/or metabolic disorders and other associated or related diseases.

In a preferred embodiment, the invention provides synergistic Annona squamosa compositions standardized to at least one acetogenin compound in the range of 0.01% to 30% comprising terminal α,β-unsaturated-γ-methyl-γ-lactone moiety as biomarker(s).

In another embodiment, the invention provides synergistic composition(s) having at least one component selected from the extract(s) or fraction(s) derived from Annona squamosa standardized to acetogenin compound(s) further contain optionally at least one component selected from pharmaceutically or dietetically acceptable excipients, vehicles, carriers and diluents or mixtures thereof for prevention, treatment, inhibition or controlling one or more inflammations and other associated or related diseases.

In another preferred embodiment, the invention provides synergistic composition(s) having at least one component selected from the extract(s) or fraction(s) derived from Annona squamosa standardized to acetogenin compound(s) in combination with Vitamins such as Vitamin C.

In yet another embodiment the synergistic compositions of the present invention are useful for the prevention, treatment, inhibition or controlling one or more inflammations and other associated or related diseases.

In yet another embodiment the synergistic compositions of the present invention are useful for the prevention, treatment, inhibition or controlling one or more of metabolic disorders and other related disorders.

In yet another embodiment the synergistic compositions of the present invention are useful for the prevention, treatment, inhibition or controlling one or more biological marker proteins associated with inflammation and other associated or related diseases.

Various embodiments relate to a composition comprising from 33% to 67% of a biologically effective extract of Annona squamosa and from 33% to 67% of Vitamin C or a pharmaceutically acceptable salt thereof, based on the combined weight of said extract of Annona squamosa and said Vitamin C;

said extract of Annona squamosa being an alcohol extract of Annona squamosa leaves comprising at least one compound selected from the group consisting of acetogenins having terminal a,b-unsaturated-g-methyl-g-lactone moiety.

Certain embodiments relate to a composition according to claim 1, comprising from 33% to 67% of a biologically effective extract of Annona squamosa and from 33% to 67% of Vitamin C or a pharmaceutically acceptable salt thereof, based on the combined weight of said extract of Annona squamosa and said Vitamin C, and further comprising at least one biologically active ingredient. The at least one biologically active ingredient may be selected from the group consisting of vitamin A, vitamin B 12, Vitamin D, vitamin E, hill oil, omega-3-fatty acid, selenium, a Boswellia serrata extract, an Andrographis paniculata extract, a Terminalia chebula extract, a Curcuma longa extract, magnesium, selenomethionine, selenocysteine, methylselenocysteine, and mixtures thereof.

### DESCRIPTION OF FIGURES

FIG. 1: Figure shows summary depiction of the bioassay guided fraction to identify most active compound and active fractions.

FIG. 2: Figure shows the chemical structures of acetogenins isolated from the extracts of the leaves of Annona squamosa.

FIG. 3: Figure shows bar diagrammatic representation of paw volumes of Freund's Complete Adjuvant induced paw edema in Sprague Dawley rats by methanol extract of Annona squamosa leaf (LI12100) at 50 mg/kg or 100 mg/kg body weight and prednisolone (10 mg/kg). The bars correspond to the paw volumes in groups treated with control, LI12100 at 50 mg/kg body weight, LI12100 at 100 mg/kg body weight and prednisolone respectively. Each bar represents mean±SD, N=6.

FIG. 4: Figure shows bar diagrammatic representation of serum TNFα concentrations in different groups of animals. After 14 days of FCA challenge, serum TNFα was quantitatively measured by enzyme-immuno assay kit (R&D Systems, USA). The bars represent the levels of the cytokines in groups supplemented with control, LI12100 at 50 mg/kg body weight, LI12100 at 100 mg/kg body weight and prednisolone (10 mg/kg) respectively. Each bar represents mean±SD, N=6.

FIG. 5: Figure shows bar diagrammatic representation of percentage inhibition of paw volumes of Freund's Complete Adjuvant induced paw edema in Sprague Dawley rats by methanol extract of Annona squamosa leaf (LI12100) at 100 mg/kg body weight, composition-1 at 100 mg/kg body weight, calcium ascorbate at 100 mg/kg body weight and prednisolone (10 mg/kg). The bars B to E correspond to the percentage inhibition of paw volumes in groups treated with LI12100, composition-1, calcium ascorbate and prednisolone respectively. Each bar represents mean±SD, N=6.

FIG. 6: Figure shows bar diagrammatic representation of serum TNFα concentrations in different groups of animals. After 14 days of FCA challenge, serum TNFα was quantitatively measured by enzyme-immuno assay kit (R&D Systems, USA). The bars A to E represent the levels of the cytokines in groups supplemented with control, LI12100 at 100 mg/kg body weight, composition-1 at 100 mg/kg body weight, calcium ascorbate at 100 mg/kg body weight and prednisolone (10 mg/kg) respectively. Each bar represents mean±SD, N=6.

FIG. 7: Modulation of metabolic markers Adipogenesis and Lipolysis processes in 3T3-L1 adipocytes by Annona squamosa leaf methanol extract (LI12100). Representative immunoblots depict down-regulation of various marker proteins such as PPARγ, ADRP, CEBPα, CEBPβ, aP2, CD36 and perilipin as indicated. The 3T3-L1 mouse pre-adipocytes were allowed to differentiate in absence or presence of 10 μg/ml or 25 μg/ml of LI12100. Vehicle control cultures received only similar concentrations of DMSO. Expression of actin protein was evaluated in each blot as the internal control. Expression of each protein was measured densitometrically and normalized with actin expression. The comparative expression levels in arbitrary units are represented as bar diagrams (side panels). The bars a, b and c represent the expressions in cells treated with vehicle control, 10 μg/ml of LI12100 and 25 μg/ml of LI12100 respectively.

FIG. 8: Bar diagram representation of % reduction in body weight in diet induced obese model of Sprague Dawley rats. The bars represent % reductions in body weight in treatment groups supplemented with LI12100 (100 mg/kg), LI12100 (250 mg/kg) and sibutramine (7 mg/kg) respectively.

FIG. 9: Bar diagrammatic representation of increase in serum adiponectin concentration in diet induced obesity model of Sprague Dawley rats. Each bar indicates mean±SD of serum adiponectin concentration at 56 days of treatment with either control or LI12100 (100 mg) or LI12100 (250 mg) as indicated in the diagram. N=7, ** indicates statistical significance (compared to the control at the end of eight week treatment; p<0.0001).

### DETAILED DESCRIPTION OF THE INVENTION

Inflammation is a response of vascular tissues to stimuli such as pathogens, damaged cells or allergic agents, which enter into the body. It is a protective mechanism in the body to remove harmful pathogens or agents and protect the tissues. Pro-inflammatory cytokines such as TNFα, IL-1β, IL-6, GM-CSF and CD4+, Th2 subset derived IL-4, IL-5 and IL-13 lymphokines are considered as the key factors of immunopathogenesis of inflammatory diseases. 5-Lipoxygenase is an enzyme critical for leukotriene synthesis from arachidonic acid, a key step in the inflammatory process. Leukotrienes are key mediators of inflammatory disease.

Metabolic syndrome is a condition involving a set of disorders that enhances the risk of heart disease. The major components of metabolic syndrome are excess weight, the cardiovascular parameters (high blood pressure, dyslipidemia, high levels of triglycerides and low levels of HDL in the blood), atherosclerosis, diabetes and insulin resistance. A subject suffering with several of these components, i.e. metabolic syndrome is highly prone to heart disease, though each component is a risk factor.

Adipocytes and macrophages play important role in the pathogenesis of metabolic syndrome and disease components associated with it. Metabolic markers, which include but not limited to PPAR-γ, Adipose Differentiation Related Protein (ADRP), CD36, Adipocyte Fatty-Acid-Binding Protein (aP2/FABP4/A-FABP), Beta-3 adrenergic receptor (β3-AR), adiponectin and Perilipin, become abnormal during obesity and metabolic syndrome and other disease conditions associated with metabolic syndrome.

Various codes of Annona squamosa extracts/fractions, Pure compound and compositions used to describe the embodiments in the specification are given below:

LI12100—Methanol extract of Annona squamosa leaf
LI12100A—Hexane extract obtained by sequential extraction of Annona squamosa leaf
LI12100B—Methanol extract obtained by sequential extraction of Annona squamosa leaf
LI12100C—Ethyl acetate extract of Annona squamosa leaf
LI12100D—Ethanol extract of Annona squamosa leaf
LI12100E—Hydroalcohol extract of Annona squamosa leaf
LI12100F—Ethyl acetate extract of Annona squamosa leaf obtained by partitioning
LI12100G—Hexane extract of Annona squamosa seed
LI12100H—Ethyl acetate extract of Annona squamosa seed
LI12100I—Mixture of Methanol extract and Water extract of Annona squamosa leaf
LI12100J—Mixture of methanol extracts of leaves and seeds of Annona squamosa
LI12101—Ethyl acetate extract obtained by partitioning of Annona squamosa leaf

The word “acetogenin” widely used in the specification and claims of the present invention, unless otherwise stated, refers to at least one acetogenin compound having terminal α,β-unsaturated-γ-methyl-γ-lactone moiety derived from Annona squamosa.

The word ‘moiety’ or ‘group’ used in the specification and claims of the present invention are interchangeable and refer to the functional group or functional moiety in the molecule.

There is ever increasing prevalence of various inflammatory diseases and metabolic disorders. The inventors have thus conducted a detailed investigation involving several in vitro and in vivo experiments on several plant extracts, fractions and pure compounds and accidentally found that administration of the extract(s) or the active fraction(s) or active compounds of the Annona squamosa or their compositions in a therapeutically effective amount in cell based studies potently ameliorated the levels of certain cytokines/chemokines/biomarkers that are over expressed during inflammation. The methanol extract (L112100) derived from the leaves of Annona squamosa potently inhibited TNFα with a half inhibitory concentration (IC₅₀) of 20.06 ng/mL. The ethyl acetate extract (LI12101) obtained through a selected process from the leaves of Annona squamosa has also shown most potent TNFα inhibition with an IC₅₀ value of 8.34 ng/mL. The other extracts (LI12100A to LI12100J) of Annona squamosa also showed potent TNFα inhibition as summarized in Table 1.

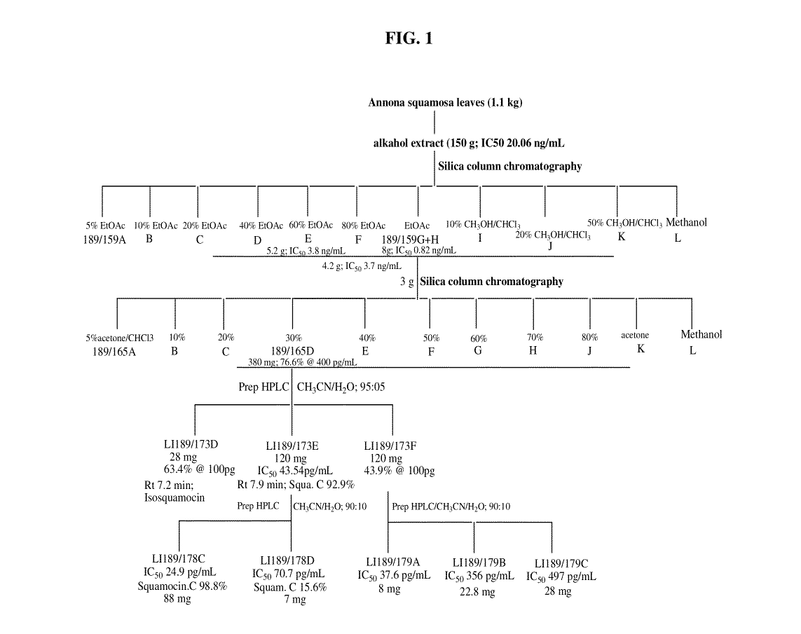

The methanol extract (LI12100) of Annona squamosa was subjected bioassay guided separation to identify the compound responsible for the TNFα inhibition. The bioassay guided purification followed by characterization of the pure compounds manifested squamocin C (LI12103) as the most potent compound with an IC₅₀ value of 24.9 pg/mL. A minor compound having 63% TNFα inhibition at 100 pg/mL was also isolated from the same fraction and identified as isosquamocin (LI12132; 2). The bioassay-guided fractionation is summarized in FIG. 1.

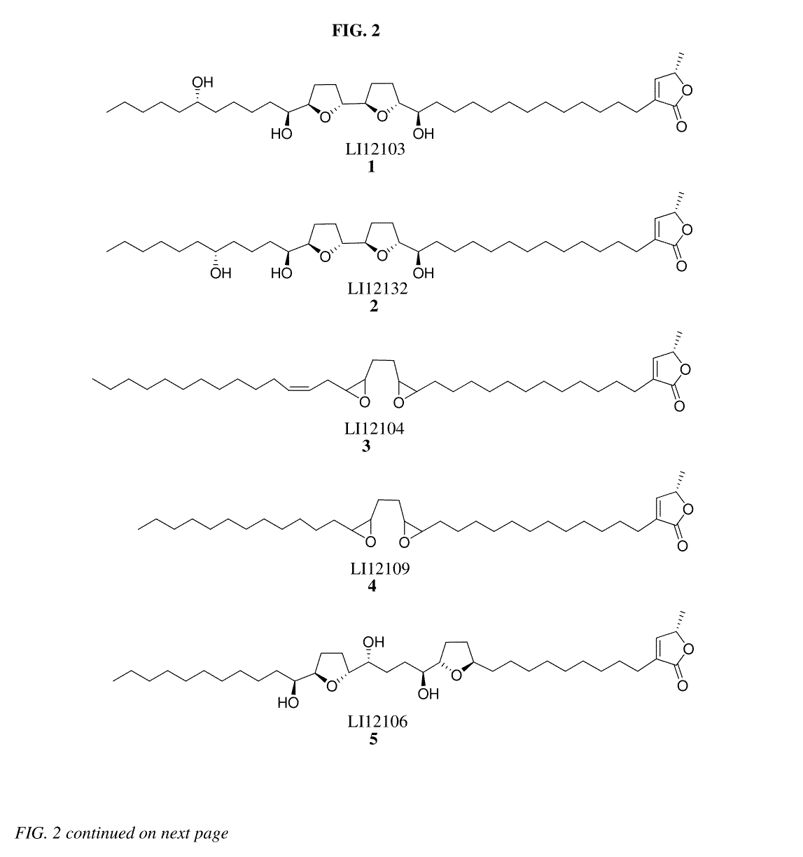

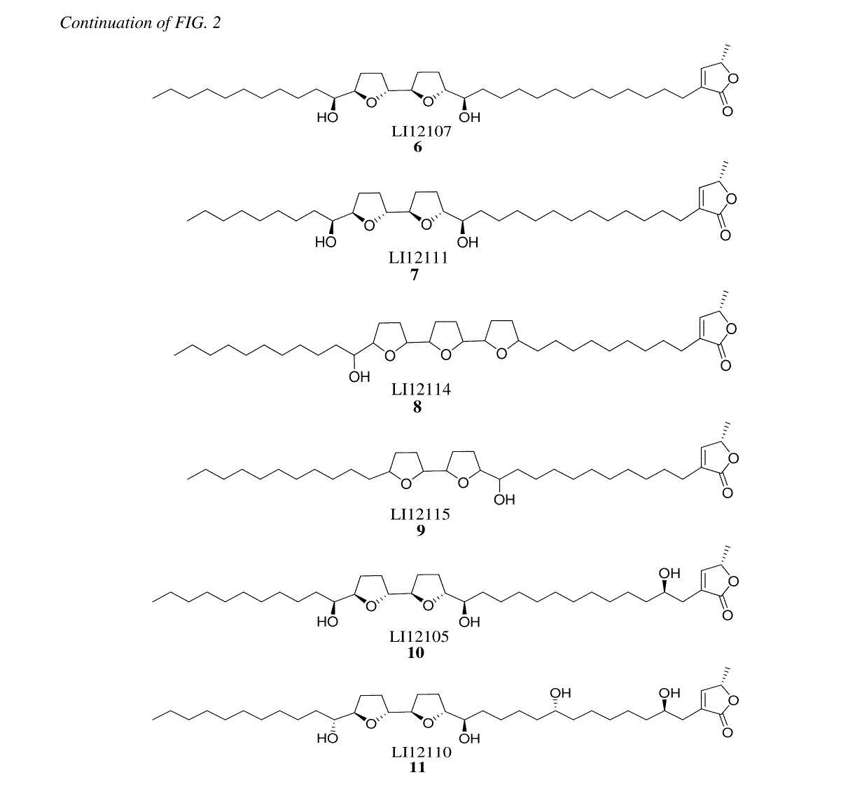

The ethyl acetate extract (LI12101) of the leaves obtained through a selected process was also subjected tedious and laborious purification process to yield closely 15 acetogenin compounds comprising terminal α,β-unsaturated-γ-methyl-γ-lactone moiety (a) having at least one functional group selected from one or more tetrahydrofuran moieties (b), one or more epoxide moieties (c), one or more hydroxyl groups, and one or more olefinic bonds (double bonds) in the alkyl chain. The compounds include squamocin C (LI12103; 1), isosquamocin (LI12132; 2), dieposabadelin (LI12109; 4), squamostatin D (LI12106; 5), squamocin L (LI12107; 6), squamocin J (LI12111; 7), squamocin G (LI12105; 10), and 10-hydroxyasimicin (LI12110; 11). The structures of compound LI12104 (3), compound LI12114 (8), compound LI12115 (9) are assigned tentatively. The structures of the known compounds and those assigned tentatively are summarized in FIG. 2.

The remaining compounds, compound LI12112, compound LI12113, compound LI12116 and compound LI12117 are also characterized to be annonaceous acetogenins with characteristic terminal α,β-unsaturated-γ-methyl-γ-lactone as per the spectral data provided in the experimental section. LI12112 is an acetogenin with characteristic terminal α,β-unsaturated-γ-methyl-γ-lactone and contains a tetrahydrofuran moiety, three hydroxyl groups and a disubstituted double bond on the alkyl chain. It has a molecular weight (MW) of 606 mass units. LI12113 is an acetogenin with molecular weight of 588 mass units and contains characteristic terminal α,β-unsaturated-γ-methyl-γ-lactone and possesses three epoxide groups on the alkyl chain. LI12116 (MW 604) is an acetogenin with characteristic terminal α,β-unsaturated-γ-methyl-γ-lactone and contains two epoxide groups and a hydroxyl group on the alkyl chain. LI12117 is an acetogenin with characteristic terminal α,β-unsaturated-γ-methyl-γ-lactone and contains two tetrahydrofuran moieties three hydroxyl groups on alkyl chain.

The biologically active acetogenin compounds of the present invention have characteristic structural features, wherein each of said acetogenin comprises a terminal α,β-unsaturated-γ-methyl-γ-lactone moiety (a). In addition the said acetogeneins comprise one or more tetrahydrofuranic (b) group(s) or one or more epoxide (c) group(s) shown below in the alkyl chain, further containing optionally one or more hydroxyl groups and/or one or more olefinic bonds (double bonds).

The biologically active acetogenin compounds of the present invention have characteristic structural features, wherein said acetogenins each comprising a terminal α,β-unsaturated-γ-methyl-γ-lactone moiety of structure (a) and an optionally hydroxylated hydrocarbon chain; said hydrocarbon chain being interrupted by at least one bivalent group selected from the group consisting of a tetrahydrofuran group of structure (b) and an epoxy group of structure (c):

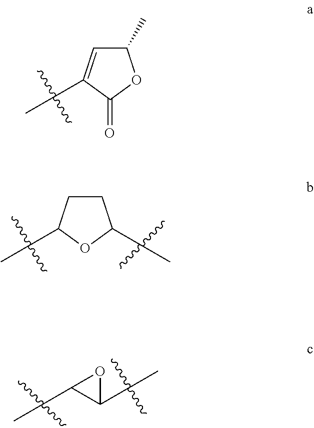

The extracts of the leaves of Annona squamosa were then standardized to squamocin C. The methanol extract (LI12100) as described above contains 0.4% of squamocin C, 0.07% of squamosin G and 0.08% of squamocin L. The ethyl acetate extract (LI12101) as described above contains 0.5% of squamocin C. The concentration of the individual acetogenins and the total concentration vary based on the nature of the raw material used for the extraction. However, an extract having as low as 0.1% of squamocin C showed potent TNFα inhibition in vitro and potent anti-inflammatory activity in vivo. The total concentration of the ‘acetogenin compounds containing ‘terminal α,β-unsaturated-γ-methyl-γ-lactone moiety’ as described above was found to be in the range of 0.2% to 5%.

The extracts potently inhibited the MMP-3 production in Interleukin-1β induced human lung tumor cell line A549 suggesting that the extracts and fractions comprising novel acetogenin composition can be useful to prevent cartilage degradation and improve joint health.

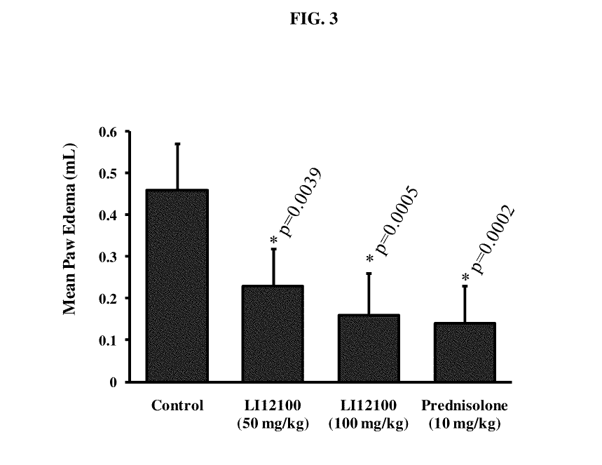

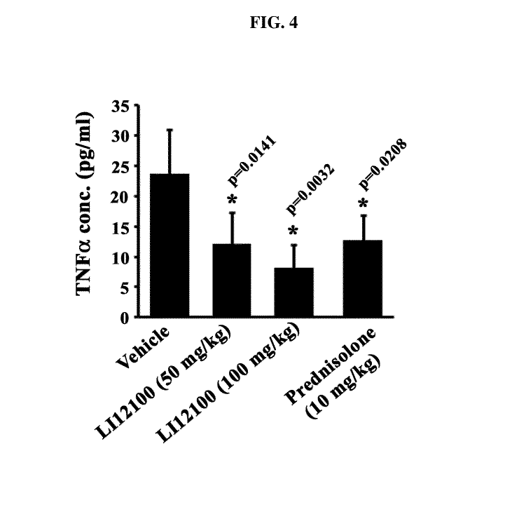

The Methanol extract of Annona squamosa leaves (1112100; standardized to 0.24% squamocin C) showed potent dose dependent efficacy in Freund's Complete Adjuvant (FCA) induced arthritis model of Sprague Dawley rats. The treatment group supplemented with LI12100 exhibited 49.5% and 64.5% reductions in paw volume at doses 50 mg/kg and 100 mg/kg body weight respectively, when compared to the control group. The positive control prednisolone (10 mg/kg body weight) exhibited 69.2% (10 mg/kg body weight) reduction in paw volume as summarized in FIG. 3. The levels of biomarker, tumor necrosis factor-alpha (TNF-α) in the serum of the treatment groups and the control group were also evaluated. The treatment group supplemented with LI12100 at both the doses showed significant reduction in the serum biomarker TNFα compared to the level exhibited by control group supplemented with 0.5% CMC as shown in FIG. 4. Further, the levels of a wide range of cytokine biomarkers were estimated in the serum of the treatment groups supplemented with LI12100 and the control group using multiplex assay (RCYTOMAG 80K) following the instructions provided by the vendor (Millipore Corporation, Billerica, Mass., USA). The treatment groups supplemented with LI12100 significantly modulated the expression of many cytokines in the serum, when compared to their respective levels exhibited by control group. The cytokines modulated by LI12100 include TNFα, IFNγ, IL-1β, IL-2, IL-4, IL-6, IL-13, MCP-1, Rantes and Eotaxin.

The foregoing discussion clearly establishes that acetogenin compound(s) having the terminal α,β-unsaturated-γ-methyl-γ-lactone moiety, the Annona squamosa derived extract(s) or fraction(s) containing acetogenin compound(s) comprising the terminal α,β-unsaturated-γ-methyl-γ-lactone moiety and their compositions are potent regulators or modulators of cytokines/chemokines or biomarker proteins such as including but not limited to TNFα, IL-1β, IL-2, IL-4, IL-6, IL-13, MCP-1, Rantes, Eotaxin, ICAM, VCAM, aP2, FLAP, CRP, CD36, 5-Lipoxygenase and MMPs and the same can be used for prevention, treatment, inhibition or controlling inflammation and disease conditions related to inflammation or immune disorders.

The other Annona squamosa derived extracts and/or fractions standardized to acetogenin(s) comprising terminal α,β-unsaturated-γ-methyl-γ-lactone moiety in combination with at least one functional group selected from tetrahydrofuran moiety or epoxide moiety or hydroxyl group or olefinic group in the alkyl chain also showed potent anti-TNFα activity (Table 1). The hexane (LI12100G) and ethyl acetate (LI12100H) extracts of the seeds of Annona squamosa also showed potent TNFα inhibition with IC₅₀ values of 3.1 ng/mL and 2.3 ng/mL respectively (Table 1).

The solvent(s) for the extraction of plants parts of Annona squamosa include but not limited to hexane, ethyl acetate, ethyl ether, chloroform, acetone, methyl isobutyl ketone (MIBK), methanol, ethanol, isopropanol, n-butanol, liquid carbon dioxide, water or mixtures thereof.

Several new compositions comprising at least one component selected from the extract(s) or fraction(s) standardized to acetogenin(s) derived from Annona squamosa comprising at least terminal α,β-unsaturated-γ-methyl-γ-lactone moiety in combination with at least one component selected from biologically active ingredient derived from plants/animals/microorganisms; pharmaceutically or dietetically acceptable active ingredients, vitamins were prepared for biological evaluation of their use in prevention, treatment, inhibition or controlling inflammation and/or immune related diseases mediated through cytokines/chemokines or other biological markers.

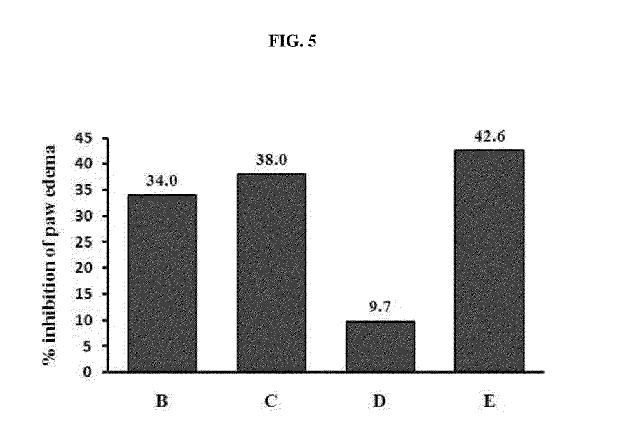

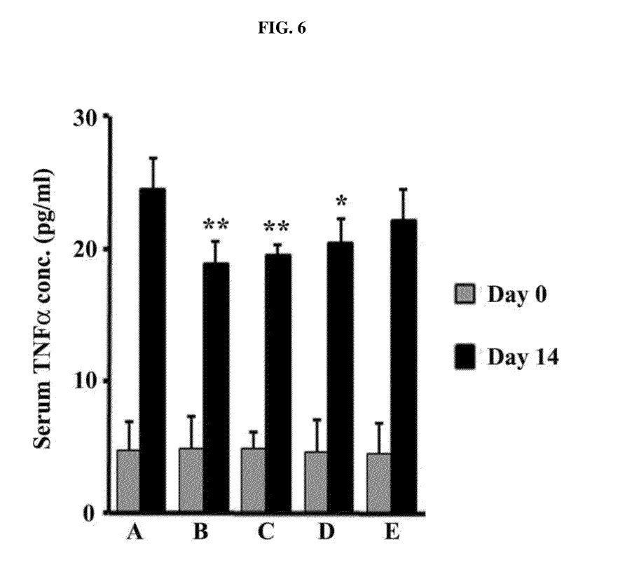

Composition-1 comprising methanol extract (LI12100) of the leaves of Annona squamosa and calcium ascorbate in 2:1 ratio was evaluated in Freund's Complete Adjuvant induced arthritis model of Sprague Dawley rats in comparison with the disease control group and positive control prednisolone group. The individual components, LI12100 and calcium ascorbate showed 34% and 9.7% reductions in paw edema respectively. However, the group supplemented with composition-1 exhibited 38% reduction in paw edema when compared to the control group as shown in FIG. 5, indicating a synergistic effect between its individual components. The treatment group supplemented with composition-1 also showed highly significant reduction in serum TNFα cytokine level as depicted in FIG. 6.

The Annona squamosa extracts comprising acetogenins were then screened for their inhibitory potential against the lipid accumulation in 3T3-L1 mouse adipocyte cells. The ethyl acetate extract (LI12101) has shown 46.6% inhibition of lipid accumulation at 10 μg/mL concentration. The other extracts also showed potent anti-adipogenesis activity as summarized in Table 4.

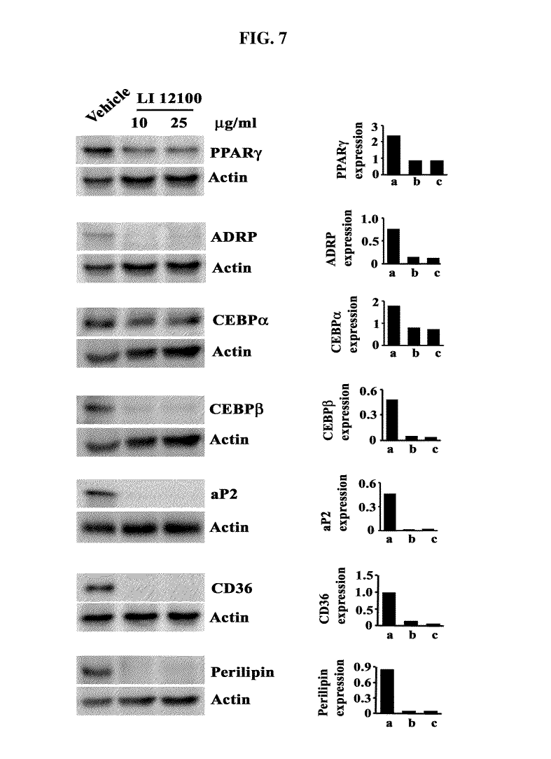

It was found that the methanol extract of Annona squamosa (LI12100) potently modulated the levels of several adipocyte differentiation markers such as Peroxisome proliferator-activated receptor gamma (PPARγ), ADRP, CEBPα, CEBPβ, CD36, Fatty Acid Binding Protein 4 (aP2/FABP4), and intracellular lipid droplet surface associated protein (perilipin) (FIG. 7) in a dose dependent manner in cellular studies performed using an immunoblot assay. The down regulation of these marker proteins in LI12100 treated adipocytes suggests that the methanol extract of Annona squamosa exerts multiple beneficial roles in controlling the adipogenic differentiation process; by (1) inhibiting cellular differentiation by down regulating PPARγ, which is a nuclear receptor protein that functions as a transcription factor for regulation of cellular differentiation, development and metabolism, (2) restricting cholesterol ester uptake by inhibiting CD36, which is a class B scavenger receptor involved in lipid uptake, (3) decreasing intracellular adiposity and intracellular lipid transport by reducing FABP4/aP2 level, which acts as a transport protein for long chain fatty acids and by (4) inhibiting adipose differentiation related protein (ADRP), which play possible role in the formation or stabilization of lipid droplets in adipocytes and enhances uptake of long chain fatty acids by adipose tissue.

Moreover, down regulation of perilipin protein in LI12100 treated adipocytes strongly indicate reduced fat store in the cytoplasm. Perilipin is a protein that coats lipid droplets in adipocytes and protect the droplets from action of hormone-sensitive lipase. Therefore it is indicative that methanol extract of Annona squamosa provides such a state where the stored lipids are more susceptible to enzymatic break down into glycerol and free fatty acids by thinning the perilipin coat around the lipid filled vesicles. In addition, LI12100 also potently down regulated the adipogenesis differentiation markers CEBPα and CEBPβ. They are proteins involved in different cellular responses like in the control of cellular proliferation, growth, differentiation and metabolism. Their down regulation by LI12100 suggests that Annona squamosa extracts could be potential agents for the prevention, treatment, inhibition or controlling metabolic diseases/disorders

Similarly, the extract LI12100 also showed potent upregulation of adiponectin protein expression in 3T3-L1 mature adipocytes. LI12100 at 5 μg/mL and 10 μg/mL showed 38% and 64% improvements in serum adiponectin concentration respectively. Adiponectin is a hormone secreted by adipocytes. It reduces intracellular triglyceride content and up-regulates glucose uptake by potentiating insulin signaling, thus it provides protection from both adipogenicity and from developing insulin resistant diabetes or type 2 diabetes. Annona squamosa extracts can thus be useful in the prevention, treatment and control of metabolic disorders through the modulation of one or more metabolic biomarkers. These non-limiting biomarkers include Peroxisome proliferator-activated receptor gamma (PPARγ), Adipose Differentiation Related Protein (ADRP), CCAAT/enhancer-binding protein alpha (CEBPα), CCAAT/enhancer-binding protein beta (CEBPβ), adipocyte CD36, Macrophage CD36, Monocyte Chemotactic protein (MCP-1), Oxidized LDL (Ox-LDL), adipocyte fatty-acid-binding protein (aP2/FABP4/A-FABP), beta-3 Adrenergic Receptor (β3AR), Perilipin, Adiponectin, Protein tyrosine phosphatase-1B (PTP-1B), Matrix Metalloproteinase-1 (MMP-1), Matrix Metalloproteinase-3 (MMP-3) and Matrix Metalloproteinase-13 (MMP-13).

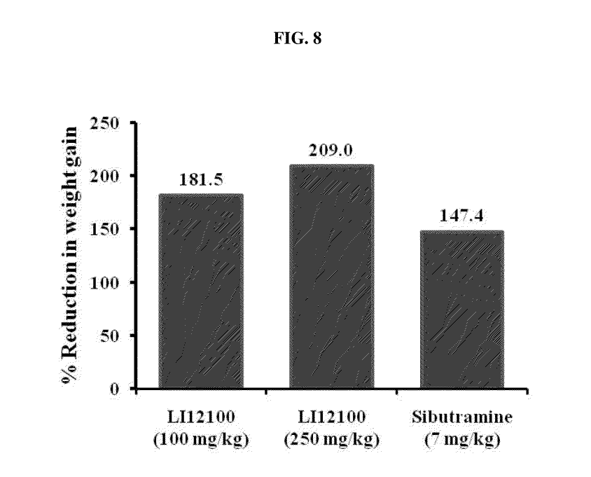

The potent anti-obesity properties shown by the extracts of Annona squamosa in in vitro models were further evaluated in an in vivo model of obesity. Obesity was induced in male Sprague Daley rats by supplementing the rats with High Fat diet for eight weeks. The rats supplemented with 100 mg/kg and 250 mg/kg body weight of LI12100 for eight weeks exhibited 181.5% and 209% reduction in body weight gain respectively when compared to the control group of animals. The positive control sibutramine at 7 mg/kg body weight showed 147.4% reduction in body weight gain compared to the vehicle treated control group. The results of body weight gain for the treatment groups and control group are summarized in FIG. 8.

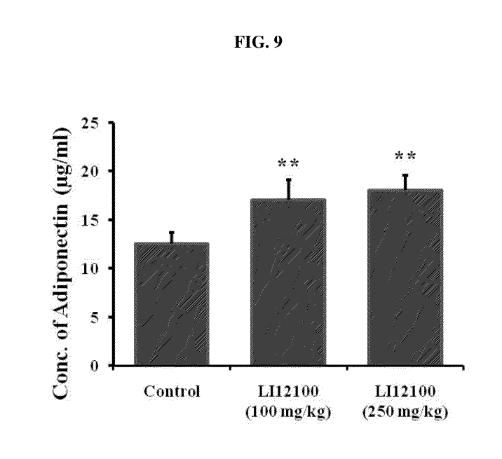

The serum adiponectin concentration was also significantly (p<0.0001) improved in the treatment group that was given a daily supplementation of LI12100 at 100 mg or 250 mg/kg body weight for 8 weeks, when compared to control group as summarized in FIG. 9. The treatment groups supplemented with 100 mg and 250 mg/kg body weight of LI12100 showed 35.9% and 43.8% improvement in serum adiponectin concentration. Hence LI12100 has potential benefit in alleviating the symptoms such as obesity, cardiovascular disorders, insulin resistant type-II diabetes, metabolic syndrome and other related disorders of metabolic syndrome.

It is obvious from this unexpected result that acetogenin(s); the extracts and fractions comprising the acetogenin(s) having α,β-unsaturated-γ-methyl-γ-lactone moiety can be used for the prevention, control and treatment of metabolic syndrome, obesity, diabetes, atherosclerosis and endothelial dysfunction and other metabolic disorders.

The compositions comprising at least one component selected from the acetogenin(s); the extracts and fractions comprising the acetogenin(s) having α,β-unsaturated-γ-methyl-γ-lactone moiety can be also be useful for the prevention, control and treatment of metabolic syndrome, obesity, diabetes, atherosclerosis, endothelial dysfunction and other metabolic disorders.

Different embodiments of the present invention are as outlined below:

In a preferred embodiment, the invention discloses synergistic compositions comprising at least one component selected from the extracts, fractions and compounds derived from Annona squamosa and at least one component selected from pharmaceutically or dietetically acceptable active ingredients, vitamins, pro-vitamins, vitamin amino acids, minerals including trace elements, essential fatty acids, fiber, various plant and herbal extracts, biologically active components” or “biologically active ingredient(s).

In the other preferred embodiment, the invention discloses synergistic compositions comprising at least one component selected from the extracts, fractions and compounds derived from Annona squamosa and at least one component selected from pharmaceutically or dietetically acceptable active ingredients, vitamins, pro-vitamins, amino acids, minerals including trace elements, essential fatty acids, fiber, various plant and herbal extracts, biologically active components” or “biologically active ingredient(s) for use in prevention, treatment, inhibition or controlling inflammation and other associated or related diseases. In the other embodiment, the extracts and fractions derived from Annona squamosa comprises at least one acetogenin compound having terminal α,β-unsaturated-γ-methyl-γ-lactone moiety.

In another embodiment, the synergistic composition comprising a biologically effective extract or fraction of Annona squamosa, wherein said biologically effective extract or fraction comprising from 0.01 to 30% by weight of at least one compound selected from the group consisting of acetogenins having terminal α,β-unsaturated-γ-methyl-γ-lactone moiety in combination.

In another embodiment, the invention provides synergistic compositions standardized to acetogenin compounds having terminal α,β-unsaturated-γ-methyl-γ-lactone moiety for the prevention, control and treatment of inflammation and other associated/related diseases.

In another embodiment, the invention provides synergistic composition(s) having at least one component selected from the extract(s) or fraction(s) derived from Annona squamosa standardized to at least one acetogenin compound containing terminal α,β-unsaturated-γ-methyl-γ-lactone moiety in combination with at least one component selected from biologically active ingredient derived from plants, animals or microorganisms.

In another embodiment, the invention provides synergistic composition(s) having at least one component selected from the extract(s) or fraction(s) derived from Annona squamosa standardized to at least one acetogenin compound containing terminal α,β-unsaturated-γ-methyl-γ-lactone moiety in combination with at least one component selected from pharmaceutically or dietetically acceptable active ingredients, vitamins, pro-vitamins, vitamin formulations, amino acids, minerals including trace elements, essential fatty acids, fiber, various plant and herbal extracts.

In other embodiment the Vitamins used for making the compositions can be selected from Vitamin A, Beta carotene, carotenoids, Vitamin B-1, Vitamin B-2, Vitamin B-3, Vitamin B-6, Vitamin B-5, Vitamin B-12, Folic acid, Biotin, Vitamin-C, Vitamin E, Vitamin-D and Vitamin-K or their salts.

Vitamin formulations can be selected from VITAMIN A (retinol, retinyl acetate, retinyl palmitate, beta-carotene); VITAMIN D (cholecalciferol, ergocalciferol; VITAMIN E-D-alpha-tocopherol, DL-alpha-tocopherol, D-alpha-tocopheryl acetate, DL-alpha-tocopheryl acetate, D-alpha-tocopheryl acid succinate); VITAMIN K [phylloquinone (phytomenadione)]; VITAMIN B1 (thiamin hydrochloride, thiamin mononitrate); VITAMIN B2 (riboflavin, riboflavin 5′-phosphate, sodium; NIACIN-nicotinic acid, nicotinamide; PANTOTHENIC ACID-D-pantothenate, calcium, D-pantothenate, sodium, dexpanthenol); VITAMIN B6 (pyridoxine hydrochloride, pyridoxine 5′-phosphate, pyridoxine dipalmitate; FOLIC ACID-pteroylmonoglutamic acid); VITAMIN B12-cyanocobalamin, hydroxocobalamin; BIOTIN-D-biotin; VITAMIN C (L-ascorbic acid, sodium-L-ascorbate, calcium-L-ascorbate, potassium-L-ascorbate, L-ascorbyl 6-palmitate).

Mineral(s) scan be selected from compounds containing calcium, chromium III metal, fluoride, iodine, iron, magnesium, molybdenum, phosphorus, potassium, selenium, sodium, sulphur and zinc.

In another embodiment, the amino acids including essential and non-essential amino acids used for making the compositions can be selected from L-Alanine, L-Ariginine, L-Asparagine, L-Aspartic acid, L-Carnitine, L-Cysteine, L-Cystine, L-Glutamine, L-Glutamic acid, L-Glycine, L-Histidine, L-Hydroxyproline, L-Isoleucine, L-Lysine, L-Methionine, L-Norleucine, L-Ornithine, L-Phenylalanine, L-Proline, L-Serine, L-Threonine, L-Tryptophan, L-Tyrosine, L-Valine, selenomethionine, selenocysteine, methylselenocysteine.

The synergistic compositions comprising Annona squamosa derived component may optionally combine with excipients, vehicles, carriers and diluents or mixtures thereof.

In another embodiment, the synergistic compositions of the present invention are useful for regulating/modulating the expression or production of one or more cytokines, chemokines and biomarkers selected from TNF-α, IL-1β, IL-2, IL-4, IL-6, IL-13, MCP-1, Rantes, Eotaxin, ICAM, VCAM, CRP, CD36, 5-Lipoxygenase and MMPs.

In another preferred embodiment, the invention provides synergistic composition(s) disclosed herein for the prevention, treatment and/or control of inflammation, wherein the inflammation and other associated and related diseases and conditions include but not limited to arthritis, asthma, atherosclerosis, endothelial dysfunction, allergic rhinitis, dermatitis, psoriasis, cystic fibrosis, inflammatory bowel diseases, interstitial cystitis, migraine, pain, angina, chronic prostatitis, sun burn, periodontal disease, multiple sclerosis, systemic lupus erythematosis, uveitis, post-angioplasty restenosis, glomerulonephritis, gastrointestinal allergies, nephritis, conjunctivitis, chronic obstructive pulmonary disease, occupational asthma, eczema, bronchitis, hay fever, hives, allergic disorders and for conditions like wheezing, dyspnea, non productive cough, chest tightness, neck muscle tightness, chest pain, joint pain and several other conditions associated thereof in mammals.

In yet another preferred embodiment, the invention further provides synergistic compositions comprising extract(s) or fraction(s) derived from Annona squamosa in combination with at least one biologically active ingredient selected from vitamin A, vitamin B 12, vitamins C, Vitamin D, vitamin E, krill oil, omega-3-fatty acid, selenium, Boswellia serrata extract standardized to Boswellic acids, Andrographis paniculata extract, Terminalia chebula extract, Curcuma longa extract, magnesium, selenomethionine, selenocysteine, methylselenocysteine or their salts and mixtures thereof for the prevention, treatment and/or control of inflammation, wherein the inflammation is selected from arthritis, asthma, atherosclerosis, endothelial dysfunction, allergic rhinitis, dermatitis, psoriasis, cystic fibrosis, Rheumatoid arthritis, Osteoarthritis, Inflammatory bowel disease, chronic obstructive pulmonary disease, allergic disorders and joint pain.

In another preferred embodiment, the non-limiting examples of arthritis as mentioned above comprise rheumatoid (such as soft-tissue rheumatism and non-articular rheumatism, fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, joint disorders, spondyloarthropathies (ankylosing spondylitis), osteoarthritis, hyperuricemia and arthritis associated with acute gout, chronic gout and systemic lupus erythematosus and degenerative arthritis.

In yet another preferred embodiment, the invention provides synergistic compositions for the modulation of the expression or production of one or more cytokines/chemokines or biomarkers/certain redox-sensitive pro-inflammatory genes related to inflammation and other associated/related diseases, wherein the biomolecules/biomarkers comprise TNFα, IL-1β, IL-2, IL-4, IL-6, IL-13, MCP-1, Rantes, Eotaxin, ICAM, VCAM, aP2, FLAP, CRP, CD36, 5-Lipoxygenase and MMPs.

In yet another embodiment, the invention also provides the method of treating inflammation, and other related diseases/disorders as disclosed herein in subjects or mammals, wherein the method comprises administering synergistic compositions as described above to the subjects or mammals in need thereof.

In another embodiment, the invention provides synergistic compositions standardized to acetogenin compound(s) comprising terminal α,β-unsaturated-γ-methyl-γ-lactone moiety for the prevention, control and treatment of metabolic disorders and other associated/related diseases.

In another embodiment, a method for altering the fat distribution in a subject is provided where the method comprises administering the synergistic composition to the subject in an amount effective to alter fat distribution in the subject. In one aspect, the alteration results from an increased metabolism of visceral fat or ectopic fat, or both in the subject. In one aspect, the methods result in a favorable fat distribution. Favorable fat distribution is an increased ratio of subcutaneous fat to visceral fat, ectopic fat, or both. In one aspect, the method involves an increase in lean body mass, for example, as a result of an increase in muscle cell mass.

In yet another embodiment, the invention provides synergistic composition for prevention, treatment, inhibition or controlling one or more metabolic disorders selected from including but not limited to obesity, overweight, diabetes, atherosclerosis, arteriosclerosis, cardiovascular diseases, hypertension, hypercholesterolemia, hyperlipidemia, hyper triglyceridemia, metabolic syndrome, endothelial dysfunction, insulin resistance, increased insulin sensitivity, hyper insulinemia, dyslipidemia, low HDL-cholesterol, lipoprotein aberrations, decreased triglycerides, elevated uric acid levels, fatty liver, non-alcoholic fatty liver disease, polycystic ovarian syndrome, haemochromatosis (iron overload), acanthosis nigricans (dark patches on the skin), impaired glucose tolerance (IGT), impaired fasting glucose (IFG), cardiovascular diseases and other metabolic disorders in warm blooded animal in need thereof.

In another embodiment, compounds useful for the treatment of various metabolic disorders, such as insulin resistance syndrome, diabetes, hyperlipidemia, fatty liver disease, cachexia, obesity, atherosclerosis and arteriosclerosis, are disclosed.

In yet another embodiment the synergistic compositions of the present invention are useful for the prevention, treatment, inhibition or controlling one or more biological marker proteins associated with metabolic disorders and other associated or related diseases, the said biological markers can be selected from PPARγ, ADRP, CEBPα, CEBPβ, CD36, aP2, perilipin and adiponectin.

In another embodiment, the invention relates to the use of the synergistic compositions containing Annona extracts and preparation of various pharmaceuticals, dietary supplements, food ingredients and beverages.

In another embodiment, the invention also provides the compositions comprising the extract and fractions derived from Annona squamosa, wherein the percentage of Annona squamosa derived component in the composition varies in the range from 0.01% to 99.9% by weight.

In another embodiment, the invention also provides the compositions comprising the extract and fractions derived from Annona squamosa, wherein the percentage of Annona squamosa derived component in the composition varies in the range from 0.1% to 50% by weight.

In another embodiment, the invention also provides the compositions comprising the extract and fractions derived from Annona squamosa, wherein the percentage of Annona squamosa derived component in the composition varies in the range from 0.1% to 30% by weight.

In the other embodiment, invention provides extract(s) and fraction(s) standardized to acetogenin(s) derived from the plant parts of Annona squamosa, wherein the plants are selected from fruits, leaves, flowers, stem, bark, root, hardwood or mixtures thereof, preferably leaves.

In another embodiment, invention provides the usage of extract(s), fraction(s), component(s) or mixtures derived from at least one herb selected from including but not limited to Commiphora mukul, Garcinia mangostana, Water melon, Amorphophallus campanulatus, Dolichos bifloras, Psidium guajava, Boswellia serrata, Curcuma longa and Terminalia chebula for preparing the compositions.

In the other embodiment, invention provides extract(s) and fraction(s) wherein the medium for extraction of the plant parts can be selected from hexane, petroleum ether, ethylether, dichloromethane, chloroform, ethyl acetate, acetone, acetonitrile, methanol, ethanol, propanol, n-butanol, iso-propanol, methyl isobutyl ketone and water or mixtures. In preferred embodiment, a few biologically active components can be selected from Glucosamine, Glucosamine salts, Chondroitin, Methylsulfonylmethane (MSM), Hyaluronic acid, collagen, polyglycans, Chitosan, Undenatured collagen type-II, SAM-e, omega-3, NEM, quercetin, boron, manganese, calcium ascorbate, flavonoids, alkaloids, phytosterols, terpenes, omega 3 fatty acid(s).

In still another embodiment, the pharmaceutically or dietetically acceptable excipients, vehicles, diluents and carriers comprises surfactants, binders, diluents, disintegrators, lubricants, preservatives, stabilizers, buffers, suspensions and drug delivery systems.

In other embodiment of the invention, the synergistic composition(s) can be formulated as oral agents such as tablets, soft capsule, hard capsule, soft gel capsules, pills, granules, powders, emulsions, suspensions, syrups, pellets, food, beverages, concentrated shots, drops and the like; and parenteral agents such as injections, intravenous drip and the like; suppositories; and transdermal agents such as patches, topical creams and gel; ophthalmic agents; nasal agents; and food or beverages.

In other embodiment, the synergistic compositions can be administered orally, topically, parenterally or by inhalation to a subject or mammal or warm blooded animal in need thereof.

In other embodiment, the synergistic composition(s) is/are administered orally, topically, parenterally or by inhalation to a subject or mammal or warm blooded animal in need thereof, wherein said ingredient or composition(s) are administered once daily or multiple administrations per day or as prescribed by physician/doctor.

In other embodiment, the synergistic compositions are delivered in the form of controlled release tablets, using controlled release polymer-based coatings by the techniques including nanotechnology, microencapsulation, colloidal carrier systems and other drug delivery systems.

In other embodiment of the invention, the synergistic compositions can be formulated into or added to existing or new food and beverage form(s) as a healthy food for warm blooded animals.

In another embodiment, the invention relates to the use of the composition(s) in preparation of various pharmaceutical dosage forms, dietary supplements, food ingredients and beverages.

In other embodiment, the extract(s) or fraction(s) derived from Annona squamosa or their composition(s) can be administered in any therapeutically effective dosage for benefits such as amelioration of symptoms, slowing of disease progression or prevention of disease at a range selected from 0.01 to 250 mg/kg body weight/day, preferably in the range from 0.1 to 50 mg/kg body weight/day.

In a further embodiment, the invention provides that therapeutically effective amount of the composition(s) can be administered in a specific dosage form such as orally, topically, transdermally, parenterally or in the form of a kit to a subject or patient in need thereof.

In accordance to the present invention, the composition of the present invention can be formulated into any dietary supplement, food and beverage forms for human and animal applications.

In another embodiment, the invention further comprises, mixing the composition of the present invention with various components used in the animal feed for the purpose of curing, preventing or treating inflammation associated or related diseases.

In our earlier Indian patent application 2526/CHE/2009 filed on 19 Oct. 2009 and corresponding PCT application # PCT/IN2010/000686 field on 19 Oct. 2010, we have disclosed Extracts, Fractions and Compositions comprising novel Acetogenins and their applications and the details are incorporated herein by reference.

The following examples, which include preferred embodiments, will serve to illustrate the practice of this invention, it being understood that the particulars shown are by way of example and for purpose of illustrative discussion of preferred embodiments of the invention and they are not to limit the scope of the invention.

### Example 1

### Preparation of Methanol Extract (LI12100) of the Leaves of Annona squamosa

Dried leaves of the plant material Annona squamosa (1.1 Kg) were pulverized to coarse powder and charged into a pilot extractor and extracted with methanol (6.6 L) at 60-65° C. temperature for 2 h. The extract was filtered and the spent raw material was re-extracted twice with methanol (2×5.5 L) under similar conditions. The combined extract was fine filtered and concentrated under vacuum to obtain methanol extract as a dark colored residue (LI12100; 150 g; 0.4% squamocin C).

### Example 2

### Preparation of Hexane Extract (LI12100A), Ethyl Acetate Extract (LI12101) and Methanol Extract (LI12100B) of the Leaves of Annona squamosa

Dried leaves of the plant material Annona squamosa (2 Kg) were pulverized to coarse powder and charged into a pilot extractor and extracted with water (14 L) at ambient temperature. The extract was filtered and the spent raw material was dried under shade. The dried spent raw material was extracted successively with cold hexane (14 L) at 10-15° C. for 2 h, followed by ethyl acetate (3×12 L) at reflux temperature for 2 h per extraction and finally with methanol (2×6 L) at 65° C. for 2 h per extraction. The extracts were fine filtered and concentrated separately under vacuum to obtain hexane extract (LI12100A; 58 g), ethyl acetate extract (LI12101; 160 g; 0.5% squamocin C) and methanol extract (LI12100B; 150 g).

### Example 3

### Preparation of Ethyl Acetate Extract (LI12100C) of the Leaves of Annona squamosa

Dried leaves of the plant material Annona squamosa (1 Kg) were pulverized to coarse powder and charged into a pilot extractor and extracted with ethyl acetate (6 L) at reflux temperature for 2 h. The extract was filtered and the spent raw material was re-extracted twice with ethyl acetate (2×6 L) under similar conditions. The combined extract was fine filtered and concentrated under vacuum to obtain a residue (LI12100C; 99 g).

### Example 4

### Preparation of Ethanol Extract (LI12100D) of the Leaves of Annona squamosa

Dried leaves of the plant material Annona squamosa (1 Kg) were pulverized to coarse powder and charged into a pilot extractor and extracted with ethanol (6 L) at 65-70° C. temperature for 2 h. The extract was filtered and the spent raw material was re-extracted twice with ethyl alcohol (2×5 L) under similar conditions. The combined extract was fine filtered and concentrated under vacuum to obtain ethanol extract as a dark colored residue (LI12100D; 132 g).

### Example 5

### Preparation of Hydroalcohol (60% ethanol) Extract (LI12100E) of the Leaves of Annona squamosa

Dried leaves of the plant material Annona squamosa (1 Kg) were pulverized to coarse powder and charged into a pilot extractor and extracted with 60% ethanol (6 L) at 65-70° C. temperature for 2 h. The extract was filtered and the spent raw material was re-extracted twice with 60% ethanol (2×5 L) under similar conditions. The combined extract was fine filtered and concentrated under vacuum to obtain hydroalcohol extract as a dark colored residue (LI12100E; 120 g).

### Example 6

### Preparation of Ethyl Acetate Partitioned Extract (LI12100F) of Leaves of Annona squamosa

The ethanol extract (25 g) as obtained in example 4 was portioned between water (200 mL) and ethyl acetate (200 mL). The organic layer was separated, dried over sodium sulfate and evaporated under vacuum to obtain a residue (LI12100F, 19 g).

### Example 7

### Preparation of Hexane Extract (LI12100G) of the Seeds of Annona squamosa

Dried seeds of Annona squamosa (300 g) were pulverized to coarse powder and extracted with hexane (1.2 L) at reflux for 2 h. The extract was filtered and the spent raw material was re-extracted twice with hexane (2×1 L) under similar conditions. The combined extract was fine filtered and concentrated under vacuum to obtain hexane extract (LI12100G; 27 g) of the seeds.

### Example 8

### Preparation of Ethyl Acetate Extract (LI12100H) of the Seeds of Annona squamosa

Dried seeds of Annona squamosa (300 g) were pulverized to coarse powder and extracted with ethyl acetate (1.2 L) at reflux for 2 h. The extract was filtered and the spent raw material was re-extracted twice with ethyl acetate (2×1 L) under similar conditions. The combined extract was fine filtered and concentrated under vacuum to obtain ethyl acetate extract (LI12100H; 27 g).

### Example 9

Compositions derived from Annona squamosa & biologically active ingredients such as Vitamins, amino acids and minerals:
1) Composition-1: Composition-1 was prepared by mixing unit doses of the following components; Two parts of Annona squamosa leaf methanol extract (LI12100) (2 g) and one part of calcium ascorbate (1 g).
2) Composition-2: Composition-2 was prepared by mixing unit doses of the following components; one part of Annona squamosa leaf methanol extract (LI12100) (1 g) and two parts of calcium ascorbate (2 g).
3) Composition-3: Composition-3 was prepared by mixing unit doses of the following components; two parts of Annona squamosa leaf methanol extract (LI12100) (2 g), two parts of Vitamin C (2 g) and two parts of Psidium guajava leaf methanol extract (2 g).
4) Composition-4: Composition-4 was prepared by mixing unit doses of the following components; Two parts of Annona squamosa leaf ethanol extract (LI12100D) (2 g) and one part of calcium ascorbate (1 g).
5) Composition-5: Composition-5 was prepared by mixing unit doses of the following components; two parts of Annona squamosa leaf methanol extract (LI12100) (2 g), one part of calcium ascorbate (1 g) and one part of Omega 3 fatty acid (1 g).
6) Composition-6: Composition-6 was prepared by mixing unit doses of the following components; one part of Annona squamosa leaf methanol extract (LI12100) (2 g) and one part of Boswellia serrata extract enriched with 30% of 3-O-acetyl-11-keto-β-Boswellic acid (AKBA) (2 g).
7) Composition-7: Composition-7 was prepared by mixing unit doses of the following components; Two parts of Annona squamosa leaf methanol extract (LI12100) (2 g), two parts of calcium ascorbate (2 g) and two parts of Boswellia serrata extract enriched with 20% of 3-O-acetyl-11-keto-β-Boswellic acid (AKBA) (2 g).
8) Composition-8: Composition-8 was prepared by mixing unit doses of the following components; one part of Annona squamosa leaf methanol extract (LI12100) (2 g) and three parts of Glucosamine hydrochloride (6 g) and one part of calcium ascorbate (2 g).
9) Composition-9: Composition-9 was prepared by mixing unit doses of the following components; one part of Annona squamosa leaf methanol extract (LI12100) (2 g), one part of Boswellia serrata extract (>10% AKBA) (2 g), two parts of Curcuma longa extract standardized 95% total curcuminoids (4 g).
10) Composition-10: Composition-10 was prepared by mixing unit doses of the following components; one part of Annona squamosa leaf methanol extract (LI12100) (2 g) and two parts of Curcuma longa extract (4 g).
11) Composition-11: Composition-11 was prepared by mixing unit doses of the following components; 2 parts of Annona squamosa leaf methanol extract (LI12100) (2 g) and one part of Vitamin E (1 g).
12) Composition-12: Composition-12 was prepared by mixing unit doses of the following components; three parts of Annona squamosa leaf methanol extract (LI12100) (3 g) and one part of Vitamin K9 (Menaquinone 9; 1 g).
13) Composition-13: Composition-13 was prepared by mixing unit doses of the following components; one part of Annona squamosa leaf methanol extract (LI12100) (1 g) and one part of Coenzyme Q10 (1 g).
14) Composition-14: Composition-14 was prepared by mixing unit doses of the following components; Two parts of Annona squamosa leaf methanol extract (LI12100) (2 g) and one part of magnesium (1 g).
15) Composition-15: Composition-15 was prepared by mixing unit doses of the following components; Two parts of Annona squamosa leaf methanol extract (LI12100) (2 g) and one part of selenomethionine (1 g).
16) Composition-16: Composition-16 was prepared by mixing unit doses of the following components; Two parts of Annona squamosa leaf methanol extract (LI12100) (2 g) and two parts of Andrographis paniculata extract (2 g).
17) Composition-17: Composition-17 was prepared by mixing unit doses of the following components; Two parts of Annona squamosa leaf methanol extract (LI12100) (2 g) and two parts of Arginine (2 g).
18) Composition-18: Composition-18 was prepared by mixing unit doses of the following components; Two parts of Annona squamosa leaf methanol extract (LI12100) (2 g) and two parts of Commiphora mukul gum resin extract (2 g).
19) Composition-19: Composition-19 was prepared by mixing unit doses of the following components; Two parts of Annona squamosa leaf methanol extract (LI12100) (2 g) and one part of Garcinia mangostana extract (1 g).
20) Composition-20: Composition-20 was prepared by mixing unit doses of the following components; One parts of Annona squamosa leaf methanol extract (LI12100) (1 g) and two parts of Water melon extract (2 g).
21) Composition-21: Composition-21 was prepared by mixing unit doses of the following components; Two parts of Annona squamosa leaf methanol extract (LI12100) (2 g) and two parts of Amorphophallus campanulatus extract (2 g).
22) Composition-22: Composition-22 was prepared by mixing unit doses of the following components; Two parts of Annona squamosa leaf methanol extract (LI12100) (2 g) and two parts of Dolichos bifloras extract (2 g).

### Example 10

### Preparation of Active Fractions and Identification of Active Compounds Using Bioassay (Anti-TNFα) Guided Purification

The methanol extract (LI12100; 150 gm; IC₅₀ 20.06 ng/mL) of Annona squamosa leaves was subjected to silica flash column chromatography using ethylacetate/hexane mixtures as eluants. The fractions eluted with 60% and 80% ethyl acetate in hexane (5.2 g and 4.2 respectively) and fraction (8 g) eluted with ethyl acetate have shown potent anti-TNFα activity. The fraction (LI189/159G+H) eluted with ethyl acetate has however shown superior activity (IC₅₀ 0.82 ng/mL). A small sample (3 g) of this fraction was subjected to further purification on silica flash column again using acetone/chloroform mixtures. The active compounds are eluted into a fraction (380 mg; 76.6% inhibition at 0.4 ng) eluted with 30% acetone/chloroform. This was further purified on HPLC using 95:5 acetonitrile/water mixture on a preparative reversed phase silica column (Phenomenex Luna 10μ, C18, 250 mm×21.2) to obtain a fraction (115 mg) having most potent activity. It was again subjected to further purification on HPLC (Phenomenex Luna 10μ, C18, 250 mm×21.2; 90:10 acetonitrile/water mixture) to obtain a pure compound (LI12103, 88 mg) having an IC₅₀ value of 24.9 pg/mL. Careful analysis of its spectral data (¹H NMR, ¹³C NMR and Mass) revealed its identity as squamocin C (LI12103; 1). A minor compound with 63% inhibition at 100 pg/mL was also isolated from the HPLC purifications and its structure is identified as isosquamocin (LI12132; 2, 28 mg). The bioassay guided fractionation is summarized in FIG. 1.

### Example 11

### Inhibition of Tumor Necrosis Factor-α (TNF-α) In Vitro by Extracts, Fractions and Compounds of Annona squamosa

The anti-inflammatory activities of extracts, fractions and compounds of Annona squamosa were assessed in a cell based in vitro assay. Briefly, THP-1 human monocytes cells were washed and re-suspended in phenol red free Dulbecco's Modified Eagle's Medium (DMEM) supplemented with 1% fetal Bovine serum (FBS). Equal number of cells was added to each well of a 96-well TC plate and the cells were pretreated for 2 h with various concentrations (ranging from 0.1 to 200 ng/mL; solutions prepared in culture medium from a stock solution containing 50 mg/l mL DMSO of each test compound) of extracts, fractions and compounds of Annona squamosa. The inflammatory response was induced by 100 ng/mL of LPS for 4 h at 37° C. in presence of 5% CO₂. The vehicle control culture wells received 0.1% DMSO in culture medium. The cell culture supernatants were collected and assessed for secretory pro-inflammatory cytokine, TNFα. The TNFα concentration was quantitatively measured by highly specific and sensitive Enzyme Immuno Assay (EIA) kit supplied by R&D Systems, USA. The enzyme immuno assay was performed based on the protocol provided by the vendor. The inhibitory concentration for 50% inhibition (IC₅₀) of TNFα was determined from a plot drawn for ingredient concentrations against corresponding TNFα levels. The IC₅₀ value for Annona squamosa extract LI12101 was found to be 8.4 ng/mL. Table 1 is a summary of 50% inhibitory (IC₅₀) concentrations of various extracts, fractions and compounds derived from Annona squamosa plant parts in cell based in vitro model.

TABLE 1

| Compound code | TNFα inhibition |
|---|---|
| or name | IC₅₀ (ng/ml) |
| LI12100 | 20.4 |
| LI12101 | 8.38 |
| LI12100A | 45.28 |
| LI12100B | 93.31 |
| LI12100C | 15.29 |
| LI12100D | 29.16 |
| LI12100E | 104.38 |
| LI12100G | 3.1 |
| LI12100H | 2.3 |
| Squamocin C | 0.0249 |
| LI12104 | >5 |
| Squamocin G | 0.124 |
| Squamostatin D | 4.08 |
| Squamocin L | 0.116 |
| Dieposabadelin | >5 |
| 10-Hydroxyasimicin | 3.21 |
| Isosquamocin | 0.080 |
| LI12112 | 3.68 |

### Example 12

### Inhibition of Matrix Metalloproteinase-3 (MMP-3) Production by the Extracts and Fractions Derived from the Leaves of Annona squamosa

Inhibition of matrix metalloproteinase-3 production by Annona squamosa extract (LI12100) was evaluated in Interleukin-1β induced human lung tumor cell line A549. Briefly, the cells were cultured in DMEM with 2 mM Glutamine, 100 U/mL penicillin, 100 μg/mL streptomycin and 10% fetal bovine serum (Hyclone, Logan, Utah). Five thousand cells per well were seeded into a 96-well cell culture plate (Corning, USA) one day before the experiment. The culture media was replaced with fresh DMEM containing 10% fetal bovine serum. LI12100 extract serially diluted in medium, ranging from 0.1 to 10 μg/mL was pre-incubated with cells for 2 hour at 5% CO2 at 37° C., and then stimulated with 10 ng/mL human IL-1β (R&D System, Minneapolis, Minn.) for 24 hours. The supernatant was harvested and used to measure MMP3 production by ELISA development kit (R&D System, Minneapolis, Minn., USA). The MMP3 concentration in culture supernatant was estimated quantitatively by interpolating the optical densities into the standard curve generated from known concentrations of MMP3. The percentage inhibition obtained at 10 μg/mL concentration for different extracts of Annona squamosa is summarized in Table 2.

TABLE 2

| Compound code | MMP3 % inhibition |
|---|---|
| or name | @ 10 μg/mL |
| LI12101 | 42.0 |
| LI12100A | 37.8 |
| LI12100B | 30.7 |
| LI12100C | 24.6 |
| LI12100D | 30.7 |

### Example 13

### The Efficacy of the Methanol Extract (LI12100) of Annona squamosa Comprising Acetogenins as Anti-Inflammatory and Anti-Cytokine Therapy

The in vivo anti-inflammatory efficacy of Annona squamosa leaf methanol extract (1112100; 0.24% squamocin C) was evaluated in Freund's Complete Adjuvant induced arthritis model of Sprague Dawley rats in comparison with positive control prednisolone. The rats of either sex were randomly selected and divided into four groups comprising 6 animals in each group. The treatment group rats were supplemented with 50 mg/kg body weight or 100 mg/kg body weight of Annona squamosa extract (LI12100) or 10 mg/kg body weight of prednisolone per day for 14 days. All the supplements were diluted in 10 mL of 1% CMC. The control group of animals was supplemented with same volume (10 mL) of 0.5% CMC. Prednisolone was administered as positive control. On the 14^{th} day, Freund's Complete Adjuvant (FCA) was subcutaneously injected in the sub-plantar region of the left hind paw of each animal. The experiment was terminated 13 days after FCA inoculation. At the end of the experiment, the animals were sacrificed, the liver tissue samples excised and stored at −80° C. Blood samples were collected from each animal at regular intervals and paw volumes were measured on the day of FCA injection and after 13 days of FCA inoculation. The difference in paw edema volume at the day of FCA injection and at 13^{th} day after FCA inoculation is considered as the inflammatory response for the supplement. The in vivo anti-inflammatory responses of Annona squamosa extract (LI12100) and prednisolone were estimated by calculating the percentage inhibition of paw edema when compared to the paw edema observed in the CMC supplemented control group. The data is summarized in FIG. 3.

The treatment groups supplemented with 50 mg/kg body weight and 100 mg/kg body weight of Annona squamosa extract (LI12100) showed 49.5% and 64.5% reductions in paw volume respectively, when compared to the control group. The positive control group prednisolone showed 69.2% reduction in the paw edema. The levels of cytokine tumor necrosis factor-alpha (TNFα) were evaluated in blood serum collected 14 days after FCA challenge. The serum cytokine levels were measured using enzyme-immune assay kit supplied by R & D systems, USA. The treatment groups supplemented with LI12100 and prednisolone showed significant reduction in serum cytokine levels (TNFα) as depicted in FIG. 4.

Further, the levels of a wide range of cytokine biomarkers were estimated in the serum of the treatment groups and the control group using multiplex assay (RCYTOMAG 80K) following the instructions provided by the vendor (Millipore Corporation, Billerica, Mass., USA). The treatment groups supplemented with LI12100 significantly ameliorated the expression of many cytokines in the serum, when compared to their respective levels exhibited by control group. The cytokines ameliorated by LI12100 include TNFα, IFNγ, IL-1β, IL-2, IL-4, IL-6, IL-13, MCP-1, Rantes and Eotaxin as summarized in Table 3.

TABLE 3
Relative expressions of cytokines/chemokines related
to inflammation in serum samples of FCA induced
Sprague Dawley rats treated with LI12100 and prednisolone

| | Relative expressions in | | | |
|---|---|---|---|---|
| Cytokines/ | Vehicle | LI12100 | LI12100 | Prednisolone |
| Chemokines | control | (50 mg/kg) | (100 mg/kg) | (10 mg/kg) |
| TNFα | 1.000 | 0.732 | 0.654 | 0.754 |
| IFNγ | 1.000 | 1.112 | 1.142 | 1.496 |
| IL-1β | 1.000 | 0.845 | 0.693 | 0.848 |
| IL-2 | 1.000 | 0.556 | 0.501 | 0.515 |
| IL-4 | 1.000 | 0.716 | 0.662 | 0.831 |
| IL-6 | 1.000 | 0.750 | 0.694 | 1.377 |
| IL-13 | 1.000 | 2.343 | 3.306 | 3.120 |
| MCP-1 | 1.000 | 0.664 | 0.701 | 0.802 |
| Rantes | 1.000 | 0.797 | 0.605 | 0.874 |
| Eotaxin | 1.000 | 0.559 | 0.484 | 0.992 |

Rat Cytokine/chemokineMultiplex panel (Catalogue No. RCYTO-80K, Millipore Corporation, USA).

### Example 14

### The Synergistic Efficacy of Composition-1 Containing the Methanol Extract (LI12100) of Annona squamosa and Calcium Ascorbate as Anti-Inflammatory and Anti-Cytokine Therapy

The in vivo anti-inflammatory efficacy of composition-1 containing the methanol extract (LI12100) of the leaf of Annona squamosa and calcium ascorbate in 2:1 was evaluated in Freund's Complete Adjuvant induced arthritis model of Sprague Dawley rats in comparison with LI12100, calcium ascorbate (CA) and positive control prednisolone. The rats of either sex were randomly selected and divided into five groups comprising 6 animals in each group. The treatment group rats were supplemented with 100 mg/kg body weight of composition-1 or 100 mg/kg body weight of Annona squamosa extract (LI12100) or 10 mg/kg body weight of prednisolone or 100 mg/kg body weight of calcium ascorbate per day for 14 days. All the supplements were diluted in 10 mL of 1% CMC. The control group of animals was supplemented with same volume (10 mL) of 0.5% CMC. On the 14^{th} day, Freund's Complete Adjuvant (FCA) was subcutaneously injected in the sub-plantar region of the left hind paw of each animal. The experiment was terminated 13 days after FCA inoculation. At the end of the experiment, the animals were sacrificed, the liver tissue samples excised and stored at −80° C. Blood samples were collected from each animal at regular intervals and paw volumes were measured on the day of FCA injection and after 13 days of FCA inoculation. The difference in paw edema volume at the day of FCA injection and at 13^{th} day after FCA inoculation is considered as the inflammatory response for the supplement. The in vivo anti-inflammatory responses of composition-1, Annona squamosa extract (LI12100), calcium ascorbate and prednisolone were estimated by calculating the percentage inhibition of paw edema when compared to the paw edema observed in the CMC supplemented control group. The data is summarized in FIG. 5.

The treatment groups supplemented with composition-1, Annona squamosa extract (LI12100) and calcium ascorbate showed 37.96%, 34.03% and 9.74% reductions in paw volume respectively, when compared to the control group. The positive control group prednisolone showed 42.56% reduction in the paw edema. The levels of cytokine tumor necrosis factor-alpha (TNFα) were evaluated in blood serum collected 14 days after FCA challenge. The serum cytokine levels were measured using enzyme-immune assay kit supplied by R & D systems, USA. The treatment groups supplemented with composition-1, LI12100 and prednisolone showed highly significant reduction in serum TNFα cytokine level as depicted in FIG. 6.

### Example 15

### Assessment of Inhibition of Lipid Accumulation in Differentiated Adipocytes by the Extracts and Fractions of Annona squamosa

One hundred thousand 3T3-L1 mouse pre-adipocyte cells in Dulbecco's Modified Eagle's Medium (DMEM) containing 10% Fetal Bovine Serum (FBS) were taken into each well of a 24-well plate and incubated for 24 h at 37° C. and 5% CO₂. Cells were pre-incubated with different concentrations of the ethyl acetate extract of Annona squamosa (LI12101) dissolved in 0.1% DMSO and then differentiated in a differentiation medium i.e. DMEM containing 500 nM insulin, 1.0 μM dexamethasone, and 0.5 mM isobutylmethylxanthine (IBMX) for 48 h. The cells incubated only with 0.1% DMSO were considered as the vehicle control. Thereafter, the differentiation medium was replaced by DMEM containing 100 nM insulin and cells in presence or absence of different concentrations of the extract LI12101 derived from Annona squamosa were incubated further for 8 days. After the treatment period, cells were fixed with 10% buffered formalin for 4 h at room temperature. The fixed cells were stained with Oil Red 0 solution (0.5 g in 100 ml isopropanol) for 10 min to measure the cellular neutral lipid accumulation. After removing the staining solution, the dye retained in the cells was eluted with isopropanol and OD was measured at 550 nm. The inhibition of fat accumulation in the treated cells was compared with the mock treated differentiated adipocytes. The anti-adipogenic activity of the Annona squamosa extract LI12101 is represented by percentage inhibition of lipid accumulation (Table 4).

The percentage inhibitions of lipid accumulation/adipogenesis caused by hexane extract (LI12100A), methanol extract (LI12100B), ethyl acetate extract (LI12100C) and ethanol extract (LI12100D) of Annona squamosa leaf were also determined using the similar protocol and data is summarized in the Table 4.

TABLE 4
Anti-adipogenic activities of the agents derived from Annona squamosa

| | Treatment | % inhibition of |
|---|---|---|
| Name of the test product | concentration | adipogenesis |
| LI12101 | 10 μg/ml | 46.6 |
| LI12100A | 10 μg/ml | 37.2 |
| LI12100B | 10 μg/ml | 21.5 |
| LI12100C | 10 μg/ml | 43.3 |
| LI12100D | 10 μg/ml | 34.1 |
| | 25 μg/ml | 41.2 |

### Example 16

### Inhibition of Adipogenesis Markers PPARγ, ADRP, CD36, aP2, CEBPα, CEBPβ, Perilipin in 3T3-L1 Adipocytes by Methanol Extract of Annona squamosa (LI12100)

Mouse pre-adipocyte 3T3-L1 cells were maintained in Dulbecco's Modified Eagles Medium (DMEM) supplemented with 2 mM glutamine, 4.5 g/L glucose and 10% fetal bovine serum. Equal number of cells was plated in each well of 24-well culture plates. Cells were pre-treated separately with 5 and 10 μg/mL of LI12100 for 2 h and followed by addition of differentiation medium containing 500 nM insulin, 1.0 μM dexamethasone, and 0.5 mM isobutylmethylxanthine (IBMX) for 48 h. Thereafter, cells were further incubated with post differentiation medium (DMEM containing 100 nM insulin) in presence or absence of LI12100. Finally, the cells were harvested, washed with chilled phosphate buffered saline and lysed with the lysis buffer. The protein extracts were clarified at 14,000 g for 20 min. Protein content was measured in Bradford method by using Coomassie blue dye and cell lysates were stored in aliquots at −80° C. until further use. The modulation of adipocyte differentiation markers such as Peroxisome proliferator-activated receptor gamma (PPARγ), Adipose Differentiation Related Protein (ADRP), CEBPα, CEBPβ, CD36, adipocyte fatty acid binding protein (aP2); and intracellular lipid droplet surface associated protein, perilipin expression were evaluated by immunoblot assay.

Inhibition of protein expression of biomarker molecules in adipocytes in the presence or absence of LI12100 was evaluated in immunoblot assay. Briefly, equal amount of cell lysate proteins were resolved in 7.5% SDS-PAGE; thereafter, the proteins were transferred to nitrocellulose membrane. After blocking the non-specific sites, the membrane was incubated with either anti-PPARγ, or anti-CD36, or anti-aP2, or anti-ADRP, or anti-CEBPα or anti-CEBPβ or anti-perilipin antibody. Finally, the specific immuno-reactive bands were developed with West-pico chemiluminescent substrate (Pierce Biotechnology, IL, USA), and the immunoblot images were recorded in a Kodak Image Station (Kodak, USA). Band intensities were calculated densitometrically and normalized with expression of actin in respective samples. The data is summarized in FIG. 7.

### Example 17

### Modulation of Adiponectin by LI12100

Modulation of adiponectin protein by LI12100 in 3T3-L1 adipocytes was evaluated in Western immunoblot assay. The cell culture, treatment protocol and immunoblot assay methodology were the same as described in Example 19. LI12100 dose dependently enhanced adiponectin protein expression in 3T3-L1 mature adipocytes. LI12100 at 5 μg/mL and 10 μg/mL showed 38% and 64% improvements in serum adiponectin concentration respectively.

### Example 18

Anti-obesity activity of Annona squamosa methanol extract (LI12100) in High Fat Diet induced obesity model of Sprague-Dawley rats.

Induction: Selected healthy Sprague-Dawley rats were randomly assigned to control or various treatment groups (n=7). All the animals allocated to the obesity study were made experimentally obese through dietary intervention during the entire eight weeks induction period by feeding high fat diet ad libitum containing Bengal gram 32 g, Wheat flour 15 g, Yeast 1 g, Butter 2 g, Ground nut oil 8 g, Casein 5 g, Vanaspathi 20 g, Vitamin mix 05 g, Milk powder 12 g and Mineral Salt mixture 4.5 g per 100 g of high fat diet.

Treatment: Following 8 weeks of induction phase, the animals were treated orally (using oral feeding gavage) with allocated test substances or vehicle daily for 8 weeks. The animals of treatment groups were supplemented with 100 mg or 250 mg/kg body weight of methanol extract of Annona squamosa leaf (LI12100) or 7 mg/kg body weight of sibutramine in 10 mL of 0.5% CMC in water for further 8 weeks. The control group of animals received only the vehicle (10 mL of 0.5% CMC in water) during this period. During the treatment phase, all animals were provided with the standard rodent diet till the end of the study.

Body weights: Body weight of individual animal was recorded weekly during the entire duration of the study. Mean body weights for the treatment group and control group were determined. The body weight gain was calculated at the end of 1st week, 4th week and 8th week after initiation of treatment in comparison to initial body weights. LI12100 dose dependently inhibited the body weight gain in high fat diet induced obese rats. LI12100 exhibited 181.5% and 209% reductions in body weight gain in the treatment groups supplemented with 100 mg/kg and 250 mg/kg body weight of LI12100 respectively. Sibutramine at a dose of 7 mg/kg exhibited 147.4% reduction in body weight gain. The results of body weight gain for the treatment groups and control group are summarized in FIG. 8.

Fat tissue weight: Abdominal and epididymal fat were isolated and weighed at the termination of the study and the results were represented in Table 5. Abdominal and epididymal fat weights in the treatment group are lower, when compared to those in the control group. The abdominal, epididymal and total fat levels were significantly reduced in the treatment group supplemented with 250 mg/kg body weight of LI12100.

Weight of fat tissues isolated from abdomen and epididymal area of rats.

TABLE 5

| Treatment | Abdominal fat (g) | Epididymal fat (g) | Total fat (g) |
|---|---|---|---|
| Control | 4.62 ± 1.93 | 4.67 ± 1.54 | 9.29 ± 3.44 |
| (10 mL/kg) | | | |
| LI12100 | 3.41 ± 1.05 | 3.95 ± 0.71 | 7.35 ± 1.68 |
| (100 mg/kg) | | | |
| LI12100 | 2.72 ± 0.68 | 3.35 ± 0.54 | 6.07 ± 1.19* |
| (250 mg/kg) | | | |
| Sibutramine | 2.68 ± 0.80 | 3.24 ± 0.89 | 5.92 ± 1.51 |
| (7 mg/kg) | | | |

Values expressed as mean weight±SD; * significant (p 0.037)

Serum Biochemistry: Blood sampling was done via sinus orbital plexus under mild anesthesia, before induction, before initiation of treatment and after completion of treatment. Various biochemical parameters including lipid profile were evaluated using biochemistry reagents supplied by Human, Germany, in an automated clinical chemistry analyzer HumaStar300, Make: Human, Germany. Mean values of the biochemical parameters especially serum cholesterol levels and triglycerides levels were estimated before induction, after induction/before treatment and after treatment. Supplementation of LI12100 at 250 mg/kg resulted in improvement in fat profile with 35.7, 43.3 and 44 percentage reductions respectively in serum cholesterol, LDL and triglycerides.

Estimation of Biomarker Adiponectin: The serum adiponectin concentration for the control and treatment groups of animals were assessed using double antibody based sandwich rat adiponectin ELISA kit. The assay was performed following the instructions provided by the manufacturer (Linco Research, USA). The sensitivity of the assay is 0.155 ng/ml. Adiponectin assay revealed that supplementation of L112100 at both 100 mg/kg and 250 mg/day/kg body weight doses showed significant improvement (p<0.0001) in serum adiponectin concentration, in comparison with the baseline. The treatment groups supplemented with 100 mg and 250 mg/kg body weight of L112100 showed 35.9% and 43.8% improvement in serum adiponectin concentration when compared to the control at 56 days of treatment. The control group, however, did not show such improvement in serum adiponectin concentration. The results are summarized in FIG. 9.

Food and water consumption were recorded daily and fasting blood samples were collected before initiation, after 4th week and 8th week (termination) of the study.

We claim:

1. Synergistic composition comprising a biologically effective extract or fraction of Annona squamosa, said biologically effective extract comprising from 0.01 to 30% by weight of at least one compound selected from the group consisting of acetogenins having terminal α,β-unsaturated-γ-methyl-γ-lactone moiety in combination with at least one biologically active ingredient selected from the group consisting of vitamin A, vitamin B 12, vitamins C, Vitamin D, vitamin E, krill oil, omega-3-fatty acid, selenium, Boswellia serrata extract standardized to Boswellic acids, Andrographis paniculata extract, Terminalia chebula extract, Curcuma longa extract, magnesium, selenomethionine, selenocysteine, methylselenocycteine and mixtures thereof.

2. The Annona squamosa extract or fraction containing acetogenins, wherein said acetogenins each comprising a terminal α,β-unsaturated-γ-methyl-γ-lactone moiety of structure (a) and an optionally hydroxylated hydrocarbon chain; said hydrocarbon chain being interrupted by at least one bivalent group selected from the group consisting of a tetrahydrofuran group of structure (b) and an epoxy group of structure (c):

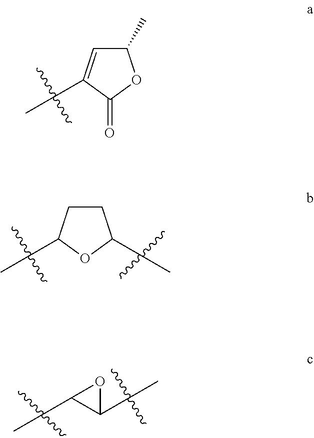

3. The herbal composition according to claim 1, wherein said acetogenins are selected from the group consisting of squamocin C (LI12103), isosquamocin (LI12132), Dieposabadelin (LI12109), squamostatin D (LI12106), squamocin L (LI12107), squamocin J (LI12111), squamocin G (LI12105), 10-hydroxyasimicin (LI12110); compound LI12104, compound LI12114, compound LI12115, compound LI12112, compound LI12113, compound LI12116 and compound LI12117.

4. The compositions as claimed claim 1, wherein the compositions further contain optionally at least one dietetically acceptable inactive ingredient selected from the group consisting of excipients, vehicles, carriers, diluents and mixtures thereof.

5. The composition according to claim 1, wherein the compositions are effective for the prevention, treatment or control of at least one condition selected from the group consisting of inflammation and diseases modulated by at least one cytokine or chemokine.

6. The composition according to 5, wherein the cytokine or chemokine is selected from the group consisting of TNFα, IL-1β, IL-2, IL-4, IL-6, IL-13, MCP-1, aP2, Rantes, Eotaxin, FLAP, ICAM, VCAM and MMPs.

7. The composition according to claim 5, wherein the disease condition modulated by at least one cytokine or chemokine is selected from arthritis, osteoarthritis, rheumatoid arthritis, asthma, atherosclerosis, endothelial dysfunction, allergic rhinitis, dermatitis, psoriasis, cystic fibrosis, inflammatory bowel diseases, conjunctivitis, chronic obstructive pulmonary disease, occupational asthma, eczema, bronchitis, allergic disorders and joint pain.

8. The composition according to claim 1, wherein the compositions are effective for the prevention, treatment or control of at least one metabolic disorder selected from the group consisting of obesity, over weight, diabetes, atherosclerosis, hypertension, hypercholesterolemia, hyperlipidemia, hypertriglyceridemia, metabolic syndrome, endothelial dysfunction, insulin resistance, increased insulin sensitivity, impaired glucose tolerance (IGT), impaired fasting glucose (IFG) or combinations there of.

9. A method of treating, inhibiting, or controlling an inflammatory disease modulated by at least one cytokine or chemokine in a mammal in need thereof, wherein the method comprises supplementing said mammal with an effective dose of a composition according to claim 1, wherein the inflammatory disease is selected from arthritis, osteoarthritis, rheumatoid arthritis, asthma, atherosclerosis, endothelial dysfunction, allergic rhinitis, dermatitis, psoriasis, cystic fibrosis, inflammatory bowel diseases, conjunctivitis, chronic obstructive pulmonary disease, occupational asthma, eczema, bronchitis, allergic disorders and joint pain.

10. A method of treating, inhibiting, or controlling a metabolic disorder in a mammal in need thereof, wherein the method comprises supplementing said mammal with an effective dose of a composition according to claim 1, where in the metabolic disorder is selected from obesity, over weight, diabetes, atherosclerosis, hypertension, hypercholesterolemia, hyperlipidemia, hypertriglyceridemia, metabolic syndrome, endothelial dysfunction, insulin resistance, increased insulin sensitivity, impaired glucose tolerance (IGT), impaired fasting glucose (IFG) or combinations thereof.

11. A composition comprising from 33% to 67% of a biologically effective extract of Annona squamosa and from 33% to 67% of Vitamin C or a pharmaceutically acceptable salt thereof, based on the combined weight of said extract of Annona squamosa and said Vitamin C;
said extract of Annona squamosa being an alcohol extract of Annona squamosa leaves comprising at least one compound selected from the group consisting of acetogenins having terminal a,b-unsaturated-g-methyl-g-lactone moiety.

12. A composition according to claim 11, further comprising:
at least one biologically active ingredient selected from the group consisting of vitamin A, vitamin B 12, Vitamin D, vitamin E, hill oil, omega-3-fatty acid, selenium, a Boswellia serrata extract, an Andrographis paniculata extract, a Terminalia chebula extract, a Curcuma longa extract, magnesium, selenomethionine, selenocysteine, methylselenocysteine, and mixtures thereof.
